(19) United States
(12) Patent Application Publication
(10) Pub. No.: US 2024/0122522 A1
(43) Pub. Date: Apr. 18, 2024

(54) METHOD FOR CHARACTERIZING ACTIVATION OF AN ANATOMICAL TISSUE SUBJECTED TO CONTRACTION

(71) Applicants: INSERM (INSTITUT NATIONAL DE LA SANTÉ ET DE LA RECHERCHE MÉDICALE), Paris (FR); UNIVERSITE CLAUDE BERNARD LYON 1, Villeurbanne (FR); CENTRE LEON BERARD, Lyon (FR); HOSPICES CIVILS DE LYON, Lyon (FR)

(72) Inventors: Cyril LAFON, Lyon Cedex 03 (FR); Jade ROBERT, Lyon (FR); Francis BESSIERE, Lyon (FR); Elodie CAO, Lyon (FR)

(21) Appl. No.: 18/569,425

(22) PCT Filed: Jun. 29, 2022
(86) PCT No.: PCT/EP2022/067830

§ 371 Date: Dec. 12, 2023

(30) Foreign Application Priority Data
Jun. 30, 2021 (EP) 21305898.5

(51) Int. Cl. A61B 5/367; A61B 5/00; A61B 5/346; A61B 8/08

(52) U.S. Cl. CPC A61B 5/367; A61B 5/346; A61B 5/7257; A61B 5/743; A61B 8/0883; A61B 8/485

(57) ABSTRACT

This disclosure relates to a method for characterizing activation of an anatomical tissue subjected to contraction, aiming at providing a characterization of the contraction itself in a quantitative manner which is accurate and repeatable. The invention proposes an analysis of the activation of the anatomical structure based on instantaneous spectral contents of an activity signal from which an identification of peaks of dominant frequency characterizing the contraction throughout the anatomical tissue results. An evolution of a spatial distribution of peaks of dominant frequency throughout the anatomical tissue over time can also be monitored. The disclosure finds particular advantageous applications in provision of a mapping of activation of the anatomical tissue in order to identify a possible dysfunction, such as an arrhythmia in a cardiac tissue.

### TECHNICAL FIELD

This disclosure relates to a method for characterizing activation of an anatomical tissue subjected to contraction.

Although not limited thereto, the disclosure specifically applies to cardiac tissue as anatomical tissue and aims at enabling provision of a mapping of activation of the anatomical tissue in order to identify a possible dysfunction, such as an arrhythmia in the cardiac tissue.

### BACKGROUND ART

Arrhythmias of the cardiac tissue are characterized by an electrical dysfunction of the cardiac cells leading to improper heartbeat. Efficient blood pumping of the normal heart is ensured by a coordinated contraction pattern of cardiac cells triggered by their electrical activation. Disturbance of this electrical activity might thus lead to contraction abnormalities and can impact the heart ability to properly complete its role. In most severe cases, especially with ventricular arrhythmias, serious functional and even life-threatening consequences might be at stake if not treated.

Radio-frequency catheter ablation (RFCA) is the clinical standard of care when an intervention is required. Currently, 3D electro-anatomical mapping is the most common interventional method used to understand the underlying mechanism occurring for each patient-specific arrhythmogenic behavior and thus guide the intervention. In focal arrhythmias especially, the exact location of arrhythmogenic foci must be detected and treated. However, this electroanatomical mapping method only provides a surface mapping of the cardiac activity, either at the endocardium or the epicardium, which sometimes is not sufficient to properly understand the ongoing mechanism in deeper layers of the tissue, and thus to ensure an efficient treatment, especially when considering ventricular arrhythmias. Mapping non-tolerated or non-sustainable arrhythmias is also challenging with the current method, as the arrhythmogenic behavior only lasts a few heartbeats, which might not be enough for proper mapping. These limitations, amongst others, imply considerable time dedicated to cardiac activity mapping and thus less time allocated to the treatment itself during electrophysiology intervention. Consequently, this could be the cause for RFCA limited efficacy, as the long term follow up demonstrated a 40% to 50% recurrence rate for ventricular arrhythmias treatment.

Novel mapping techniques are today under investigation to address this limitation. Electrocardiographic imaging (ECGI) is based on reconstruction of epicardial electrical activity from noninvasive high-density body-surface potentials recordings. ECGI requires precise registration with a 3D geometrical scan and lacks accuracy, especially in the presence of scar tissue. Techniques based on cardiac MRI have also shown promising results for tissue characterization in electrophysiology. But these methods are only limited to substrate analysis and depict late gadolinium enhancement distribution.

In the past decades, intracardiac echocardiography has been integrated in electrophysiology procedures to improve treatment guidance and efficacy while possibly reduce patient exposure time to radiations, by providing high-resolution and real-time imaging of the cardiac anatomy. In the last few years, increase in acquisition frame rate capacity with compounding methods has allowed development of novel ultrasound-based methods to map short-lived event occurring during cardiac activation. Using an acoustic approach during electrophysiology intervention is of great interest as it would offer the possibility to map cardiac activity into the myocardium, which is not feasible with the standard 3D electroanatomical mapping. Tissue Doppler Imaging (TDI) and Clutter Filter Wave Imaging (CFWI) are ultrasound-based methods which can be used for cardiac activity characterization. TDI is already available on clinical scanner but is angle-dependent and requires high computational costs. CFWI is based on selective high pass filtering of tissue velocities and provides higher signal-to-noise ratio and lower computational costs than TDI. Cardiac activation is then visualized from the motion of the attenuated velocity band. Visualization of EW propagation thus requires prior knowledge of the velocities that are to be attenuated along with user selection of the velocity threshold for filtering. Ultrafast acoustoelectric imaging (UAI) is also an ultrasound-based method developed to measure cardiac activation. UAI combines ultrasound emissions and invasive electrical recordings to map cardiac current densities and has been used on isolated rat hearts and in-vivo swine model.

Electromechanical Wave Imaging (EWI) relies on mechanical mapping of the cardiac activity derived from ultrafast ultrasound acquisitions. EWI allows tracking of local contraction propagation induced by electrical activation: the Electromechanical Wave (EW). Strain-based EWI was shown to accurately describe cardiac activation on in-vivo animal model. It also has been validated against electrophysiological measurements during sinus and paced rhythms. Angle independency was also demonstrated on healthy patients. Strain-based EWI analysis also allowed identifying the region of early activation in clinical studies.

Using strain-based EWI however implies derivation of displacement data which might be highly noisy, especially in in-vivo conditions. Representation of cardiac activation from strain-based EWI also involves manual selection of zero-crossing of hundreds of strain curves, which is time consuming and might lead to inter-operator variability.

Early stages of EWI development were based on the analysis of interframe tissue displacement, from which interframe strain is derived. Contraction propagation into the heart was then successfully visualized based on temporal evolution of myocardium displacement. EWI based on interframe displacement cineloops also allowed to accurately discriminate endocardial from epicardial pacing but also from sinus rhythm in a previous study on a working heart model (F. BESSIERE et al., “High Frame Rate Ultrasound for Electromechanical Wave Imaging to Differentiate Endocardial From Epicardial Myocardial Activation”, Ultrasound Med. Biol., vol. 46, no. 2, pp. 405-414, February 2020).

This known method of EWI based on interframe displacement cineloops enable visualizing movements of the cardiac tissue and analyzing effects of contraction in a qualitative manner. It however requires trained reader for cineloops interpretation. Interpretation could be even more challenging on more complex datasets, such as in in-vivo conditions.

### SUMMARY

This disclosure improves the situation.

It is proposed a method for characterizing activation of an anatomical tissue subjected to contraction, in particular a cardiac tissue, the method comprising:
acquiring an activation signal representative of an electrical activity of the anatomical tissue with respect to time, and determining an activation time period including a single electrical pulse corresponding to the contraction,
in synchronization with acquisition of the activation signal, acquiring consecutive images of the anatomical tissue at a high cadence over the activation time period, and segmenting each image of the anatomical tissue in a plurality of pixels,
for each pixel, determining an activity signal representative of a mechanical activity of the pixel with respect to time between consecutive images over the activation time period,
for each pixel, dividing the activation time period in a plurality of elementary time windows, calculating a spectral content of the activity signal in each elementary time window, determining a dominant frequency in each elementary time window and identifying a peak of dominant frequency in the activation time period, the peak of dominant frequency characterizing the contraction.

Hence, the invention proposes an analysis of the activation of the anatomical structure based on instantaneous spectral contents of the activity signal from which the identification of peaks of dominant frequency throughout the anatomical tissue results. This results in a characterization of the contraction itself in a quantitative manner which is more objective and repeatable.

The method may further comprise:
for each pixel, determining a contraction timing corresponding to a pixel peak time at which the peak of dominant frequency occurs from an initial time of the activation time period,
displaying a pattern of contraction by attributing a display parameter to the contraction timing of each pixel.
   In doing so, an evolution of a spatial distribution of the peaks of dominant frequency throughout the anatomical tissue over time can be monitored.

Identifying the peak of dominant frequency in the activation time period may comprise:
determining a tissue peak time at which peaks of dominant frequency are reached in a largest number of pixels comprised in the tissue,
refining the activation time period with respect to the tissue peak time, and
defining the peak of dominant frequency for each pixel as a first local maximum of dominant frequency within the refined activation time period.

In particular, refining the activation time period may comprise beginning the activation time period at a time interval before the tissue peak time, in particular, in case of cardiac tissue, the time interval corresponding to a duration of isovolumetric contraction of the cardiac tissue.

Determining the activity signal may comprise measuring a mechanical parameter representative of the mechanical activity of each pixel on the images.

In particular, the mechanical parameter may be chosen among a displacement of the pixel and a strain of the pixel.

The high cadence may be N images per second and the activation time period Ta may be divided in successive elementary time windows Tf such that Tf is between Ta/3 and Ta/12 and two successive elementary time windows are shifted of at most 0.5*N·Tf images, preferably 0.25*N·Tf images, more preferably 0.1*N·Tf images, with one another, N being preferably greater than or equal to 500 images per second, preferably N≥1000 images per second, preferably N≥1500 images per second and more preferably N≥2000 images per second, and in particular, in case of cardiac tissue, Ta being between 30 ms and 120 ms.

Calculating the spectral content of the activity signal in each elementary time window may be performed by implementing Short Time Fourier transform.

Acquiring images may be performed by ultrasound modality, images of a plane in tissue thickness being acquired.

The ultrasound modality may be implemented in electromechanical wave imaging.

Acquiring images may be performed by an intracorporeal ultrasound probe configured to emit an ultrasound signal and to receive echoes of a reflected signal.

The disclosure also relates to a method for detecting a dysfunction of the anatomical tissue, in particular an arrhythmia of the cardiac tissue, method for detecting comprising:
implementing the method for characterizing previously defined in relation with a displaying of a pattern of contraction, and
detecting the dysfunction based on an evolution of a spatial distribution of the peaks of dominant frequency throughout the anatomical tissue over time.

### BRIEF DESCRIPTION OF DRAWINGS

Other features, details and advantages will be shown in the following detailed description and on the figures, on which:

FIG. 1 is an flowchart of a method for characterizing activation of an anatomical tissue subjected to contraction according to an embodiment of the invention,

FIG. 2 is a schematic representation of a step of acquiring images of a cardiac tissue according to an example of implementation of the method of FIG. 1, showing a) an ultrasound probe arranged into the right atria from femoral vein and b) the ultrasound probe arranged into the left ventricle from the aorta,

FIG. 3 is a schematic representation of different acquisition protocols of the step of acquiring images of FIG. 2, showing a) the cardiac tissue stimulated by an activated pacing electrode arranged within a field of view of the ultrasound probe, b) the cardiac tissue stimulated by an activated pacing electrode arranged outside the field of view of the ultrasound probe and c) the cardiac tissue in sinus rhythm,

FIG. 4 is a representation of a step of segmenting the images of the cardiac tissue according to the example of implementation of the method of FIG. 1,

FIG. 5 is a representation of a step of determining an activation time period according to the example of implementation of the method of FIG. 1,

FIG. 6 is a representation of a step of determining an activity signal over the activation time period according to the example of implementation of the method of FIG. 1,

FIG. 7 is a representation of a step of calculating a spectral content of the activity signal in each of elementary time windows of the activation time period according to the example of implementation of the method of FIG. 1,

FIG. 8 is a representation of a step of determining a dominant frequency in each elementary time window according to the example of implementation of the method of FIG. 1.

FIGS. 9 and 10 are representation of steps of identifying a peak of dominant frequency characterizing the contraction according to the example of implementation of the method of FIG. 1, the peak of dominant frequency being defined as a first local maximum of dominant frequency within a refined activation time period,

FIG. 11 is a representation of a step of displaying a pattern of contraction according to the example of implementation of the method of FIG. 1,

FIG. 12 illustrates a comparison of time evolution of displacement of the cardiac tissue and dominant frequency at different timings during a period of local electrical activity, showing a) inter-frame displacement of the cardiac tissue overlaid on B-mode images, b) local electrical activity recorded simultaneously and c) the time evolution of the dominant frequency distribution,

FIG. 13 illustrates cardiac activation obtained for consecutives acquisitions during a) sinus rhythm or b) paced rhythm with pacing electrode into the field of view of the ultrasound probe.

### DESCRIPTION OF EMBODIMENTS

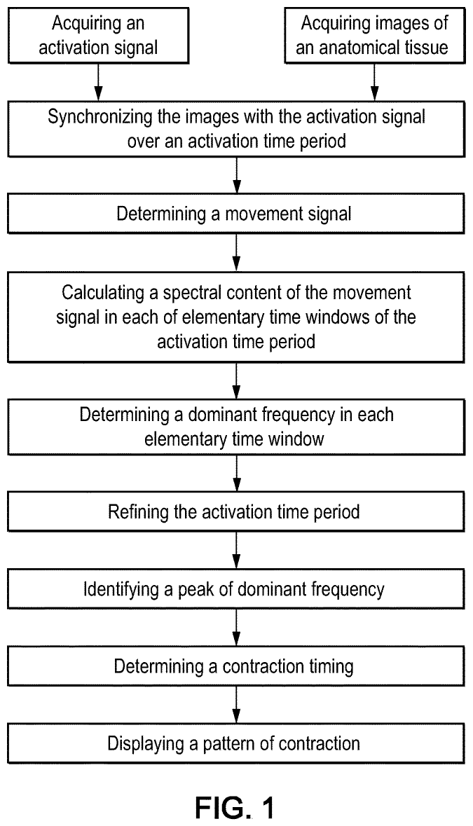

FIG. 1 is a flowchart illustrating an embodiment of a method for characterizing activation of an anatomical tissue subjected to contraction. According to a possible implementation, the anatomical tissue is a cardiac tissue of a living being, in particular a patient.

The method comprises a step of acquiring an activation signal representative of an electrical activity of the anatomical tissue with respect to time. Such activation signal may be acquired in any suitable manner, such as an electrocardiogram. The acquisition can be performed in locally at vicinity of the anatomical tissue under study or remotely, in which case a distance, and hence corresponding time offset, between a location of acquisition and a location of processing shall be taken into account.

Based on an evolution of the electrical activity over time, an activation time period including a single electrical pulse corresponding to the contraction can be determined.

The method comprises a step of acquiring consecutive images of the anatomical tissue at a high cadence of N images per second. The acquisition of the images in made in synchronization with the acquisition of the activation signal. A high cadence can be understood as a cadence such as N greater than or equal to 500 images, preferably N≥1000 images, preferably N≥1500 images and more preferably N≥2000 images. To that end, any imaging modality enabling such high cadence to be reached can be implemented. In a particularly suitable example, an ultrasound modality is implemented in electromechanical wave imaging and images of a plane in tissue thickness are acquired. Advantageously, the images are acquired internally with respect to the living being body, by an intracorporeal ultrasound probe configured to emit an ultrasound signal and to receive echoes of a reflected signal. Alternatively, the images could be acquired externally with respect to the living being body by an extracorporeal ultrasound probe.

The acquired images are then registered with the activation signal over the activation time period.

Each acquired image is segmented, manually or in an automated manner, to identify pixels belonging to the anatomical tissue. For each pixel of the anatomical tissue, an activity signal representative of a mechanical activity of the pixel with respect to time between consecutive images over the activation time period is determined. To that end, a mechanical parameter representative of the mechanical activity of each pixel is measured on the successive images to define an intra-frame displacement or an intra-frame strain for each pixel. Any suitable mechanical parameter, such as a displacement, a strain, a propagation velocity of shear waves or other, could be implemented.

The method then comprises a step of calculating a spectral content of the activity signal in each of elementary time windows of the activation time period of each pixel. The activation time period Ta may be divided in successive elementary time windows Tf such that Tf is between Ta/3 and Ta/12 and two successive elementary time windows are shifted of at most 0.5*N·Tf images, preferably 0.25*N·Tf images, more preferably 0.1*N·Tf images with one another to overlap with respectively at least 50%, at least 75% and at least 90% of consecutive elementary time windows. In the case of cardiac tissue where isovolumetric contraction, namely the contraction only due to the electrical pulse of activation, is of the order of magnitude of 30 ms, Ta can be chosen between 30 ms and 120 ms. The spectral content of the activity signal in each elementary time window may be performed by implementing Short Time Fourier transform.

From the spectral content of each pixel, a dominant frequency in each elementary time window can be determined.

In case several dominant frequencies are present in the activation time period, a tissue peak time at which peaks of dominant frequency are reached in a largest number of pixels comprised in the tissue can be determined, for example by plotting an histogram. Based on the determined tissue peak time, the activation time period can be refined by defining an initial time of the activation time period at a time interval before the tissue peak time. The time interval corresponds to a duration of isovolumetric contraction of the anatomical tissue, in the order of magnitude of 30 ms in the case cardiac tissue as mentioned previously. Any other suitable method to discriminate the peak of dominant frequency between several dominant frequencies could however be implemented. For example, one or several features of the peak of dominant frequency, such as a duration, a shape, an amplitude or other, could defined and used to discriminate this peak of dominant frequency from other dominant frequencies. Methods ensuring 2D continuity could also be implemented. Alternatively, the peak of dominant frequency could be discriminated “manually” by an operator.

The method comprises a step of defining a peak of dominant frequency characterizing the contraction for each pixel in the activation time period as a first local maximum of dominant frequency within the refined activation time period.

To provide an exploitable mapping of the contraction of the anatomical tissue, the method further comprises:
for each pixel, determining a contraction timing corresponding to a pixel peak time at which the peak of dominant frequency occurs from the initial time of the activation time period,
displaying a pattern of contraction by attributing a display parameter, such as a color, an intensity, a grey level or other, to the contraction timing of each pixel.

The aforementioned method may be implemented in a method for detecting a dysfunction of the anatomical tissue, in particular an arrhythmia of the cardiac tissue, the dysfunction being determined based on an evolution of a spatial distribution of the peaks of dominant frequency throughout the anatomical structure.

### Example

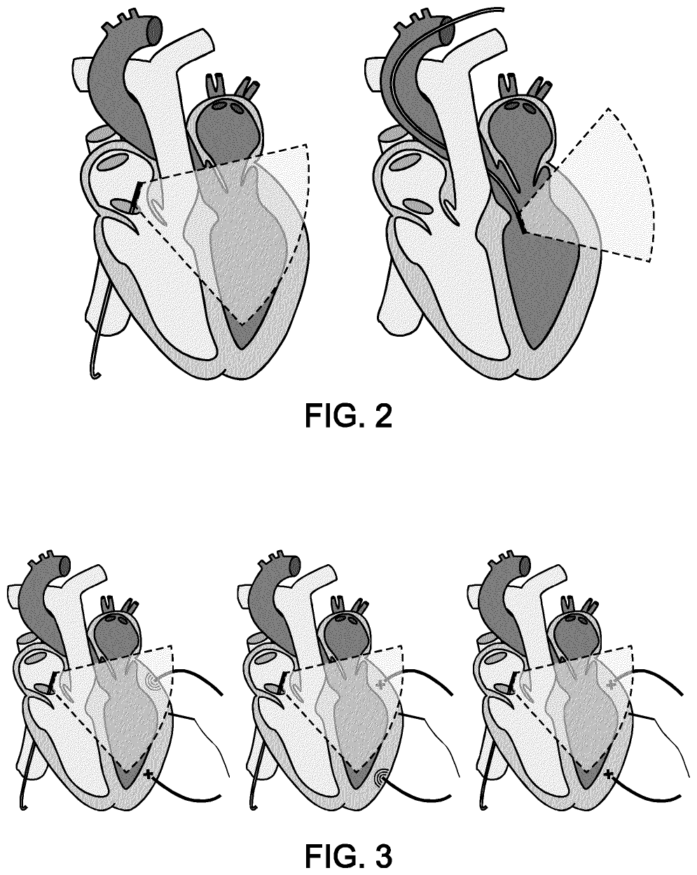

The above disclosed method is implemented in the present example disclosed for a purely illustrative and non-limitative purpose in relation with FIGS. 2 to 13.

Materials and Methods

Experimental Protocol

Animal Model

A study protocol was approved by the local Animal Research Ethics Committee (CEEA50) in accordance with recommendations of the Directive 2010/63/EU of the European Parliament on the protection of animals used for scientific purposes. Pigs (Large White Landrace, 40 kg, n=2) were pre-medicated with an intramuscular injection of ketamine (10 mg/kg to 20 mg/kg), acepromazine (0.1 mg/kg) and buprenorphine (9 μg/kg). Anesthesia was induced using an intravenous propofol bolus of 1 mg/kg to 2 mg/kg and maintained with isoflurane and ventilation (air/oxygen 50/50) was ensured after intubation. ECG, arterial pressure, temperature as well as oxygen saturation were monitored throughout the experiment. An anti-arrhythmic protocol was also set up with continuous perfusion of lidocaine (7 mg/kg/hour to 10 mg/kg/hour), amiodarone (1 mg/kg/hour to 2 mg/kg/hour) and magnesium sulfate (20 mg/kg/hour). Sternotomy was finally performed to expose the heart for leads placement.

Ultrasound Probe

A 64-elements intracardiac echocardiographic (ICE) probe (Vermon, France) with a 6.25 MHz central frequency, integrated in a 9F catheter, was used.

FIG. 2 shows ICE probe insertion. Two different accesses were used to achieve left ventricle lateral or anterior wall imaging. The ICE probe was inserted either into the femoral vein and mounted up to the right atria or the right ventricle, or inserted directly into the left ventricle (LV) from the aorta. For the inferior vena cava through femoral access, a vascular sheath was used to guide and stabilize the ICE probe position, which was not feasible for the aortic access, because of the too large diameter of the sheath. Fluoroscopy and B-mode imaging were both used to guide ICE probe placement. An imaging field of view (FOV) of the ICE probe was a 60° sector with approximatively 6 cm depth to acquire data into the lateral or anterior wall of the LV.

Pacing and Electrical Recording Leads Placement

For each imaging position of the ICE probe, pacemaker leads (Tendril 52 cm, Abbott, Minneapolis, MN, USA) were screwed at the epicardium of the LV wall to induce arrhythmic behavior. At least one lead was positioned in the imaging FOV guided by B-mode imaging of the ICE probe itself. A second lead was positioned at another position of the ventricle wall, outside the imaging plane.

An example of lead placement for ultrasound acquisitions protocol is depicted in FIG. 3. For five imaging planes studied, five consecutive ultrafast ultrasound acquisitions were performed by stimulating the heart using successively the pacing electrode screwed in the imaging FOV (left), the one screwed outside the imaging FOV (middle) and during sinus rhythm without pacing (right).

Each lead was successively connected via a wire to a device controller (Merlin, Abbott, Minneapolis, MN, USA) for every imaging plane. Pacing parameters were settled 0.2 V above the threshold to ensure a local capture that would not jeopardize activation mapping within the LV wall. The duration of the stimulus was set at 1 ms. Sites were consecutively paced at least 20 bpm above the spontaneous sinus rhythm. Two electrodes were fixed at the surface of the heart, as also shown in FIG. 3, to acquire a local bipolar signal using differential amplifier (DAM 50, Word Precision Instrument, Sarasota, FL, USA) which was recorded using an oscilloscope (PicoScope 3000, Pico Technology, St. Neots, UK).

Ultrasound Acquisition In-Vivo

All ultrafast ultrasound acquisitions were achieved with a programmable research scanner (Vantage, Verasonics, Kirkland, Washington, USA). Acquisitions were performed at three positions of the ICE probe for the first swine, including a section of the LV anterior or lateral wall. For the first two positions (P1 and P2), ICE probe was inserted directly into the LV from the aorta. For the third position (P3), ICE probe was inserted into the right atria from the femoral vein. For the second swine, acquisitions were performed along two positions, both with ICE probe inserted from the femoral vein into the right atria for the first one (P4-5) or into the right ventricle for the second one (P6). For position P4-5, two pacing electrodes were screwed into the section of the ventricle wall being imaged in the current FOV. For all the other positions (P1, P2, P3 and P6), only one pacing electrode was included into the FOV.

For each imaging plane, acquisitions triggered by the local electrical recordings were performed five times in sinus rhythm (without pacing) and five times for each pacing electrode (15 acquisitions per imaging plane). As there were two pacing electrodes into the FOV of P4-5, acquisitions in sinus rhythm or by pacing from the electrode outside the FOV were repeated ten times. To achieve ultrafast acquisitions, compounded diverging waves at 5 angles (−20°, −10°, 0°, 10°, 20°) were used. Acquisition frame rate was set at 2500 fps, after compounding, with an imaging depth of 220λ (5.4 cm) for P1 and P2 and at 2000 fps for P3, P4-5 and P6 with imaging depth set at 260λ (6.6 cm).

In summary, amongst the 90 acquisitions performed, 30 were achieved while pacing with the electrode included in the imaging FOV, 30 while pacing from outside the FOV and 30 in sinus rhythm, as summarized in Table I.

TABLE I

| ICE PROBE | | | | | |
|---|---|---|---|---|---|
| POSITION | P1 | P2 | P3 | P4-5 | P6 |
| ICE insertion | Aorta | Aorta | Inferior | Inferior | Inferior |
| access | | | vena | vena | vena |
| | | | cava | cava | cava |
| LV region imaged | Anterior | Lateral | Lateral | Lateral | Anterior |
| | wall | wall | wall | wall | wall |
| Acquisition frame | 2500 | 2500 | 2000 | 2000 | 2000 |
| rate (fps) | | | | | |
| Imaging depth | 220/54 | 220/54 | 250/62 | 260/64 | 270/66 |
| (λ/mm) | | | | | |
| Nb. of acquisitions | 5/5/5 | 5/5/5 | 5/5/5 | 10/10 | 5/5/5 |
| in sinus rhythm/in | | | | (5 for each | |
| FOV/outside FOV | | | | pacing lead)/ | |
| | | | | 10 | |

Acquisitions were synchronized with local electrical activity through bipolar recordings.

Data Processing

Cardiac Tissue Segmentation

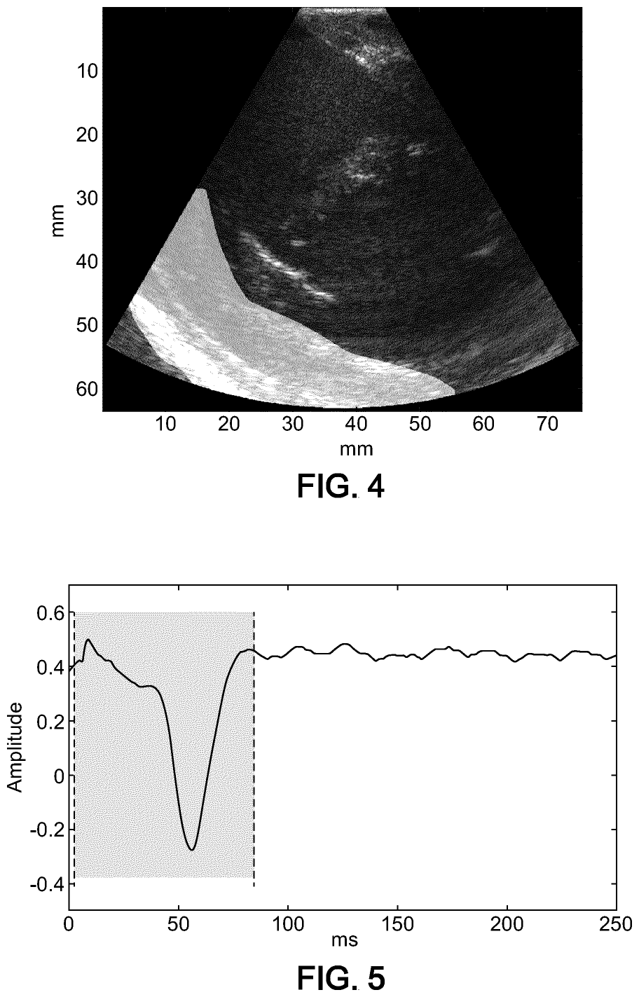

On FIG. 4, to define the region of interest, B-mode cineloops were reconstructed from IQ data at each acquired frames. Square-root compression and 8-fold numerical gain was applied on the absolute value of the IQ data. Manual segmentation was then performed for each reconstructed B-mode dataset at a frame within the electrical activity time window for which the imaging plane was stable (no large movement of the cardiac tissue).

Interframe Displacement Reconstruction

From the raw IQ data, axial inter-frame displacement maps were computed using phase-tracking algorithm and were registered with ECG recordings. Time-window of interest was manually defined for each acquisition by selecting the activation time period with the pulse of local electrical activity on the ECG recordings. Cineloops of tissue displacement was analyzed to determine the first region undergoing contraction.

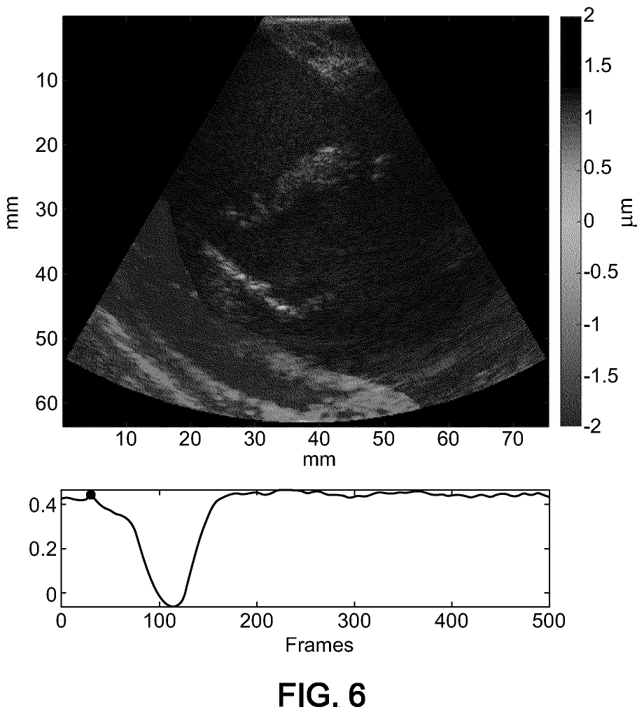

FIG. 5 illustrates acquired frames of ultrasound data time-registered with the electrical signal recorded locally on the LV epicardium and period of electrical activity is manually delimited. And FIG. 6 shows the local displacement of cardiac tissue computed from IQ data between each pair of consecutive frames with a phase-tracking algorithm.

Short Time Fourier Transform of Interframe Displacement

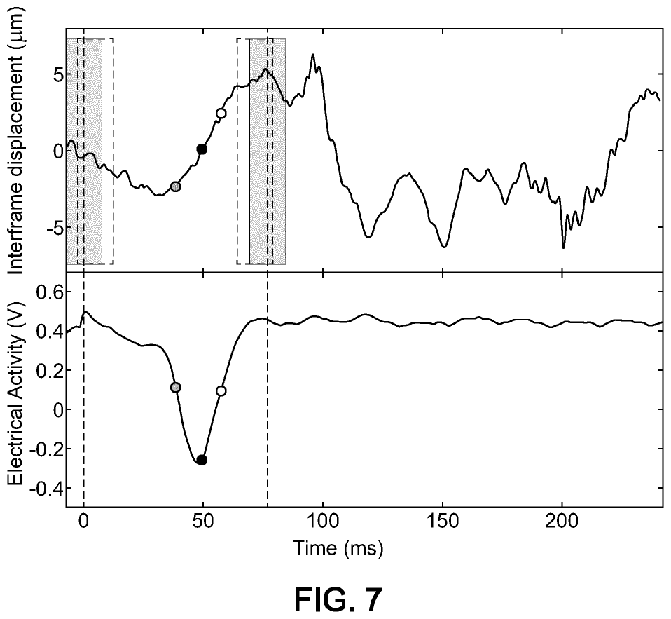

For each pixel of cardiac tissue in the imaging plane, time evolution of the instantaneous spectral content of its displacement was assessed, using Short Time Fourier Transform (STFT). STFT of the activity signal based on 1 D-displacement was performed over a sliding elementary time window of 30 frames (namely 12 ms for P1 and P2, and 15 ms for P3, P4-5 and P6). In other words, for each pixel, the activity signal corresponding to an interframe displacement was divided into equal, overlapping elementary time window. Fourier Transform was then applied to each segment of the activity signal within one of the elementary time windows, separately. The first elementary time window for STFT was centered at the local onset of electrical activity, as manually determined on the ECG recordings. The elementary time window was then repeatedly shifted by 2 frames (=0.8 ms for P1 and P2 and =1 ms for P3, P4-5 and P6, overlap 93%) until reaching the end of the activation signal of electrical activity, as depicted in FIG. 7.

Time Evolution of Dominant Frequency

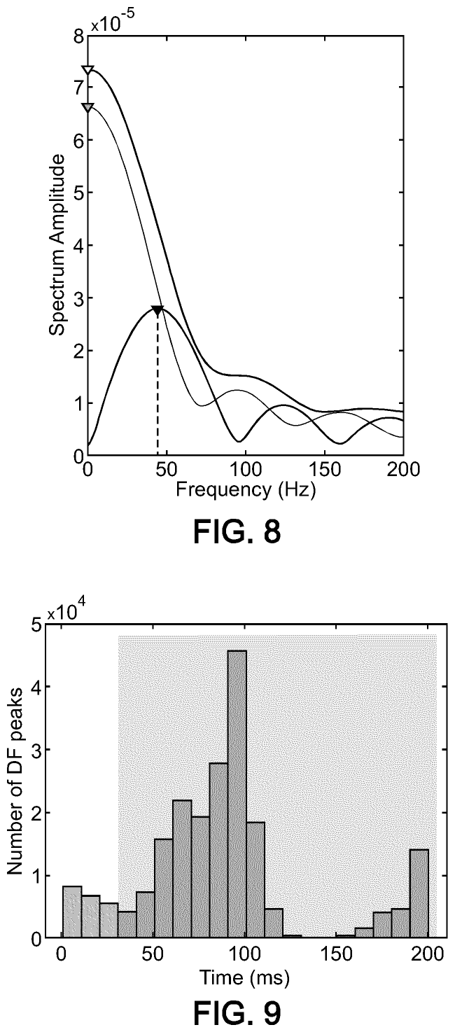

At each elementary time window, the spectral content or frequency spectrum of the activity signal segment was computed. For a given elementary time window, dominant frequencies (DF) were retrieved: each pixel was attributed with the frequency with highest amplitude in the spectrum as illustrated in FIG. 8. For each pixel, one could thus plot the variation of the STFT dominant frequency with the position of the elementary time window. This curve illustrates the variation of the instantaneous dominant frequency depending on the chosen segment of the displacement curve, as shown in FIGS. 7 and 8. It was thus considered that electromechanical wave propagation at a given pixel was indicated by a shift in the dominant frequency as the cardiac tissue starts displacing when undergoing contraction. For each pixel, time evolution of dominant frequency is computed by retrieving the frequency with maximum amplitude in the spectrum for each position of the window.

Refining Time Period of Interest Via DF Peaks Histogram

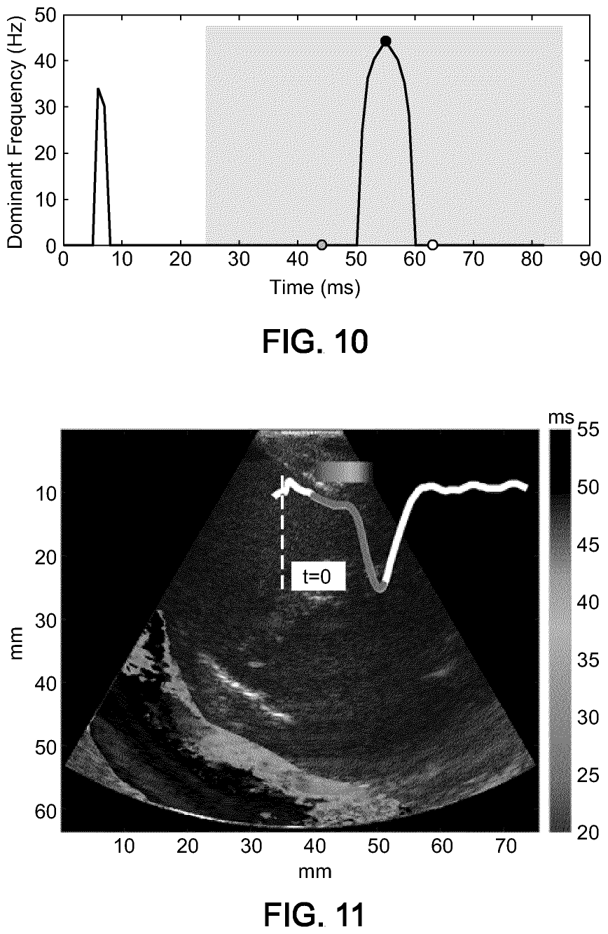

For some pixels, several dominant frequency shifts were observed, as illustrated in FIG. 10. Another processing step thus had to be implemented to refine the activation time period of interest during which DF shift would actually be attributed to cardiac contraction. To define the final activation time period of interest, histograms of peaks of dominant frequency were plotted for all pixels of the cardiac tissue. Bin with highest amplitude was considered as the tissue peak time at which the majority of the tissue was undergoing contraction, as shown in FIG. 9. Electromechanical wave was indeed considered propagating into the cardiac tissue of the FOV when the majority of the pixels was experiencing a DF shift. The final activation time period of interest was set 60 frames before this timing, to be able to visualize the initiation of the cardiac contraction. In other words, the peak of dominant frequency of a given pixel was considered as representative of contraction only when occurring 24 ms (for P1 and P2) or 30 ms (for P3, P4-5 and P6) before the timing of contraction of the majority of the cardiac tissue, at earliest. This step was required to avoid selecting DF shifts which were occurring before the electromechanical wave, caused by noise or other mechanical waves for example.

2D Cardiac Activation Map: First DF Peak Timing Isochrone

Finally, 2D activation maps were computed from these DF curves, into the defined time period of interest. The first timing at which the DF reached a peak was defined as the contraction timing at a given pixel. The pattern of contraction could then be displayed by attributing a display parameter to the contraction timing of each pixel. On FIG. 11, 2D activation maps were then obtained by plotting contraction timing isochrones. Time origin for these isochrones was defined as the onset of local electrical activity as defined on the ECG recordings. Cardiac tissues with earliest contraction are indicated by red pixels or pixels with a first grey level whereas tissues with latest contraction are indicated by blue pixels or pixels with a second grey level.

Results

EWI Cineloop and Activation Isochrone

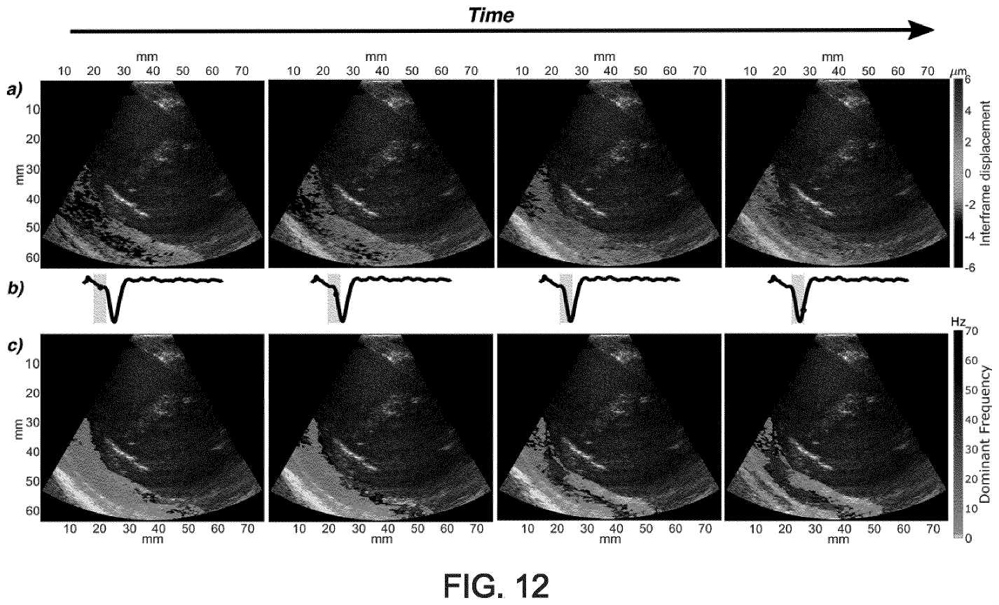

FIG. 12 shows a comparison of time evolution of tissue displacement and dominant frequency at different timings during the period of local electrical activity with:
a) interframe displacement of the cardiac wall overlaid on B-mode image,
b) local electrical activity recorded simultaneously with the ultrasound data, the timing at which the corresponding interframe displacement was computed being indicated with a dot, and the position of the elementary time window for Short Time Fourier Transform of activity signal, and thus for generating the corresponding dominant frequency map, being indicated in light blue, and
c) 2D representation of the dominant frequency distribution time evolution providing indication as to pixels with 0 Hz dominant frequency at the given elementary time-window and showing a propagation pattern of positive interframe displacement similar to the propagation pattern of non-zero dominant frequencies, both indicative of the electromechanical wave propagation.

FIG. 12 a) shows interframe displacement of the cardiac tissue during sinus rhythm at different timing with ICE probe in position P3. Movement towards the probe, upwards, and movement downwards are displayed with different display parameter such as color or grey level. Electromechanical wave propagation can be tracked by the transition of displacement direction of the contracting tissue from one display parameter to another. For this specific case, first occurrence of electromechanical wave appears at the endocardium and propagates towards the epicardium of the ventricle wall.

The same propagation pattern can be seen when considering the DF at different time windows, as shown in FIG. 12 c). At the beginning of the contraction, the majority of the cardiac tissue has a 0 Hz instantaneous dominant frequency, as the tissue is quasi-static. When contraction starts at the endocardium, higher DF values appear locally and then also propagate towards epicardium.

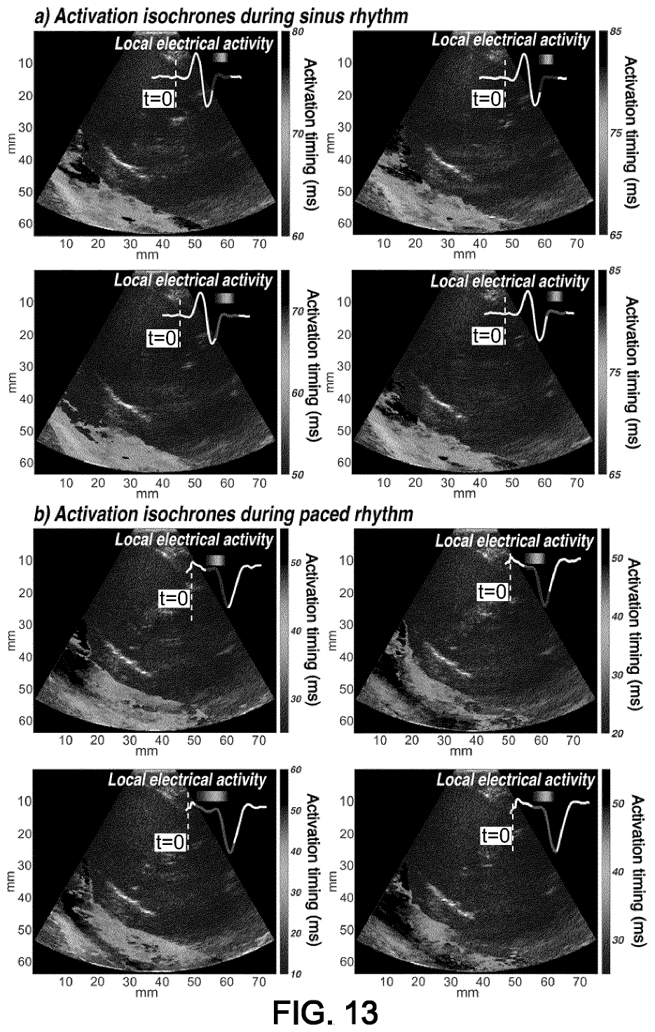

If the timing at which each pixel reaches its peak of DF is now considered as the contraction timing, electromechanical wave propagation can be represented through a contraction isochrone, as depicted in FIG. 13 c). Regions reaching a DF peak, and thus contracting, earlier and regions reaching their DF peak, and thus contracting, later being represented with different display parameters. It can be noticed that the propagation pattern obtained by the analysis of the time evolution of the instantaneous spectral content of the interframe displacement is similar to the one that can be visualized using displacement cineloops.

Amongst the 75 remaining acquisitions, 61 (81%) continuous contraction isochrones were automatically obtained, the 14 other isochrones were discontinuous and could thus not be representative of an electromechanical wave propagation. “Holes” in isochrones indicate regions where no DF peak was detected within the final activation time period of interest.

Activation Isochrones for Consecutive Heartbeats

On FIG. 13 a), isochrones obtained at 4 consecutive acquisitions during sinus rhythm at position P3 are represented. Electromechanical wave propagation pattern is highly qualitatively similar across all five acquisitions. The contraction delay respective with the electrical activity onset is highly repeatable for these acquisitions. For all acquisitions, the onset of the contraction is observed at end of the second half of the period of local electrical activity.

On FIG. 13 b), isochrones obtained for 4 consecutive acquisitions while pacing the heart with an electrode in the imaging plane at the same ICE probe position are displayed. In this case, 4 isochrones depict realistic EW front propagation with a continuous pattern. Once again, even in paced rhythm, contraction patterns depicted by the isochrones are repeatable. The synchronicity between electrical and mechanical activation is less repeatable than during sinus rhythm but contraction seem to appear overall earlier with respect to local electrical activity onset.

### DISCUSSION

Intracardiac EWI Feasibility

75 EWI acquisitions were achieved with sufficient imaging plane stability and interframe displacement of cardiac tissue allowed visualization of contraction front propagation. It was demonstrated that contraction isochrones were representative of the cardiac electro-mechanical activation, as it matched the EW propagation pattern previously visualized with time evolution of interframe displacement maps of the cardiac tissue (F. BESSIERE et al., “High Frame Rate Ultrasound for Electromechanical Wave Imaging to Differentiate Endocardial From Epicardial Myocardial Activation”, Ultrasound Med. Biol., vol. 46, no. 2, pp. 405-414, February 2020).

Analysis of Cardiac Tissue Interframe Displacement Instantaneous Frequency Content Evolution to Generate Contraction Isochrones.

In total, 75 acquisitions were achieved with fair stability of the imaging plane and were analyzed using a novel algorithm based on SIFT of the interframe displacement. This analysis relies on the shift to higher frequencies of the instantaneous frequency content of displacement signal when EW is propagating in the cardiac tissue. Electromechanical wave propagation in the cardiac tissue induces a local tissue displacement correlated with a local electrical activity. Just before contraction, at the end of ventricular diastole, the cavity is completely filled with blood and the cardiac tissue reaches a quasi-static state and is thus motionless. At early ventricular systole, isovolumetric contraction of the cardiac wall occurs as the electromechanical wave propagates. Cardiac cells are indeed generating local displacement of the tissue, indicative of its contraction, which leads to shifting of the instantaneous frequency content from a 0 value (as the tissue was not moving, just before contraction) to a higher value. During this period, no large amplitude displacement of the tissue is observed, and the cardiac tissue is only locally displacing as a result of the contraction. Based on this analysis, continuous isochrones depicting realistic EW front propagation were obtained in 81% of the cases.

1. Method for characterizing activation of an anatomical tissue subjected to contraction, the method comprising:
acquiring an activation signal representative of an electrical activity of the anatomical tissue with respect to time, and determining an activation time period including a single electrical pulse corresponding to the contraction,
in synchronization with acquisition of the activation signal, acquiring consecutive images of the anatomical tissue at a high cadence over the activation time period, and segmenting each image of the anatomical tissue in a plurality of pixels,
for each pixel, determining an activity signal representative of a mechanical activity of the pixel with respect to time between consecutive images over the activation time period,
for each pixel, dividing the activation time period in a plurality of elementary time windows, calculating a spectral content of the activity signal in each elementary time window, determining a dominant frequency in each elementary time window and identifying a peak of dominant frequency in the activation time period, the peak of dominant frequency characterizing the contraction.

2. Method according to claim 1, further comprising:
for each pixel, determining a contraction timing corresponding to a pixel peak time at which the peak of dominant frequency occurs from an initial time of the activation time period,
displaying a pattern of contraction by attributing a display parameter to the contraction timing of each pixel.

3. Method according to claim 1, wherein identifying the peak of dominant frequency in the activation time period comprises:
determining a tissue peak time at which peaks of dominant frequency are reached in a largest number of pixels comprised in the tissue,
refining the activation time period with respect to the tissue peak time, and
defining the peak of dominant frequency for each pixel as a first local maximum of dominant frequency within the refined activation time period.

4. Method according to claim 3, wherein refining the activation time period comprises beginning the activation time period at a time interval before the tissue peak time.

5. Method according to claim 1, wherein determining the activity signal comprises measuring a mechanical parameter representative of the mechanical activity of each pixel on each consecutive images.

6. Method according to claim 5, wherein the mechanical parameter is chosen among a displacement of the pixel and a strain of the pixel.

7. Method according to claim 1, wherein the high cadence is N images per second and the activation time period Ta is divided in successive elementary time windows Tf such that Tf is between Ta/3 and Ta/12 and two successive elementary time windows are shifted of at most 0.5*N·Tf images, with one another, N being greater than or equal to 500 images.

8. Method according to claim 1, wherein calculating the spectral content of the activity signal in each elementary time window is performed by implementing Short Time Fourier transform.

9. Method according to claim 1, wherein acquiring images is performed by ultrasound modality, images of a plane in tissue thickness being acquired.

10. Method according to claim 9, wherein the ultrasound modality is implemented in electromechanical wave imaging.

11. Method according to claim 9, wherein acquiring images is performed by an intracorporeal ultrasound configured to emit an ultrasound signal and to receive echoes of a reflected signal.

12. The method according to claim 1, wherein the anatomical tissue is cardiac tissue.

13. Method according to claim 12, wherein refining the activation time period comprises beginning the activation time period at a time interval corresponding to a duration of isovolumetric contraction of the cardiac tissue.

14. Method according to claim 7, wherein two successive elementary time windows are shifted of at most 0.5*N·Tf images, or 0.25*N·Tf images, or 0.1*N·Tf images, with one another, and wherein N≥1000 images, or N≥1500 images or N≥2000 images.

15. The method according to claim 7, wherein the anatomical tissue is cardiac tissue and the Ta is between 30 ms and 120 ms.
